(19) United States
(12) Patent Application Publication
(10) Pub. No.: US 2015/0208692 A1
(43) Pub. Date: Jul. 30, 2015

(54) ADDITIVE FOR ANIMAL FOOD OR DRINKING WATER FOR RUMINANTS

(71) Applicant: KLAUS NEUFELD, HEILIGENKREUZ (AT)

(72) Inventor: KLAUS NEUFELD, HEILIGENKREUZ (AT)

(21) Appl. No.: 14/624,027

(22) Filed: Feb. 17, 2015

Related U.S. Application Data
(63) Continuation-in-part of application No. PCT/EP2013/066782, filed on Aug. 13, 2013.

(30) Foreign Application Priority Data
Aug. 14, 2012 (AT) A888/2012
Aug. 20, 2012 (AT) A904/2012

(51) Int. Cl. A23K 1/16; A61K 31/352

(52) U.S. Cl. CPC A23K 1/1618; A61K 31/352

(57) ABSTRACT

An animal feed additive contains oligomeric procyanidins for the treatment or prophylaxis of acute or subacute ruminal acidosis or conditions resulting from ruminal acidosis in a ruminant.

### CROSS-REFERENCE TO RELATED APPLICATION

This application is a continuation-in-part (CIP), under 35 U.S.C. §120, of copending international application No. PCT/EP2013/066872, filed Aug. 13, 2013, which designated the United States; this application also claims the priority, under 35 U.S.C. §119, of Austrian patent applications A 888/2012, filed Aug. 14, 2012 and A 904/2012, filed Aug. 20, 2012; the prior applications are herewith incorporated by reference in their entirety.

### BACKGROUND OF THE INVENTION

### Field of the Invention

The invention relates to additives to animal feed or drinking water for ruminants.

Nowadays, the aim of farm animal production is to obtain an acceptable yield in the livestock under conditions which are as economical as possible and to obtain a high state of health. A good state of health has been shown to result in good yields as regards growth, milk production, wool production, reproduction, etc. Attempts have previously been made to increase the yield by so-called nutritional feeding of antimicrobial substances (for example antibiotics). In respect of ruminants, the product Flavomycin® (substance name: flavophospholipol) has been used for a long time [Van der Merwe B J et al: The effect of flavophospholipol (Flavomycin®) on milk production and milk urea nitrogen concentrations of grazing dairy cows, South African Journal of Animal Science (SASAS) 2001, 31(2): 101-105]. A further example for this group of substances is that of ionophore antibiotics, for example the product Rumensin (substance name: Monensin-sodium) [Callaway T R et al: Ionophores: Their use as ruminant growth promotants and impact on food safety, Curr. Issues Intest. Microbiol. 2003, 4: 43-51], but in the meantime this has been banned in the EU as an animal feed additive for ruminants. Monensin is obtained from Streptomyces cinnamonensis. In the context of the widespread earlier use of Monensin in ruminants, it has been shown that, in addition to a positive influence on the activity of the ruminal flora, Monensin has a positive effect on the prevalence of ruminal acidosis. This influence has been attributed to the fact that Monensin prevents excessive carbohydrate conversion and thus prevents over-acidifying the rumen by influencing the activity of the ruminal flora (“damping effect” on the microbial flora). This effect has also been detected with other antimicrobial substances. Anti-microbial enzymes are not stated to exhibit a direct influence on carbohydrate-cleaving enzymes.

The use of antimicrobial substances is linked to significant disadvantages. On the one hand, from a scientific viewpoint, a positive effect of these substances on the yield of the livestock has not always been shown [Gritzer K and Leitgeb R: Überprüfung der Wirksamkeit antibiotischer and mikrobieller Leistungsförderer in der Rindermast [Examination of the effectiveness of antibiotic and microbial yield promotants in beef cattle], Die Bodenkultur, 1998, 49(1): 51-59], but on the other hand, among consumers, there is now a trend towards natural and residue-free animal food products, and thus commercial and consumer resistance has developed to the use of such substances in agriculture. In addition, in scientific circles, the suspicion has arisen that the use of antibiotic yield promotants in agriculture can result in the development of resistance to antibiotics, in particular in addition to their use in human medicine. In recent years, so-called live yeasts have started to be used as alternative products. They are known to promote bacteria in the rumen and to have a stabilizing effect on the pH in the rumen due to better lactic acid conversion [Schmitz W: Lebendhefen: Kleine Zellen mit groβer Wirkung [Live yeasts: small cells with a large effect], Der fortschrittliche Landwirt 2010, 20: 10-12].

However, in veterinary medicine, it has been shown that the greatest capacity for optimal yield in agricultural livestock is obtaining an optimal state of health. In ruminant agricultural livestock for example cattle, sheep or goats, there are two major metabolic problems which lead to substantial drops in yield. These are on the one hand ketosis, and on the other hand acidosis, in particular subacute acidosis (SARA). Ketosis and acidosis are of approximately equal economic significance.

Subacute ruminal acidosis (SARA) occurs at a frequency of 10% to 30 in dairy cows, depending on the district and mode of farming being investigated. Thus, Enemark and Jorgensen (2001) estimated a frequency of 22% for dairy cows in Denmark, while Oetzel (2003) estimated the frequency of subclinical ruminal acidosis in early lactating cows to be 15%. Thus, acidosis is more prevalent than ketosis and thus constitutes the most important health problem on economic grounds for cattle production.

Previous methods for preventing ruminal acidosis are of limited efficacy. Normally, buffer substances, in particular sodium bicarbonate, are added to the animal feed. However, in effective doses of >150 g per animal per day, sodium bicarbonate leads to a reduced animal feed uptake and thus can only be used in a limited manner.

The use of acarbose and trestatins for the production of a drug for the curative, palliative and prophylactic treatment of ruminal acidosis is known from U.S. Pat. No. 7,022,684 B2 and its counterpart European published patent application EP 1 157 696.

In international (PCT) patent application publication WO 2011/127499, I describe an animal feed additive which contains a lignocellulose-containing, iron ion-binding material such as wood, wood fibers, bark or bark fibers from species of pine, in combination with a vegetable antimicrobial substance such as an extract or plant material from plants of the genus Magnolia or the cinnamomum plant genus. The action of antimicrobial substances such as magnolol and honokiol is optimized by combination with the iron ion-binding components, since the iron ion-binding components reduce the availability of the iron, whereupon bacteria of the ruminal flora which require iron for growth become more sensitive to the action of the antimicrobial substances.

### SUMMARY OF THE INVENTION

The present invention aims to provide means for the treatment or prophylaxis of acute or subacute ruminal acidosis or conditions resulting from ruminal acidosis in ruminants. These means comprise an animal feed, an animal feed additive or a premix for the manufacture of an animal feed, as well as a drinking water additive.

In accordance with one aspect, the present invention provides an animal feed additive which contains oligomeric procyanidins for the treatment or prophylaxis of acute or subacute ruminal acidosis or conditions resulting from ruminal acidosis in ruminants.

In accordance with one aspect of the invention, the oligomeric procyanidins in the animal feed additive may be in the form of ground pine bark, ground elm bark, pine bark extract, elm bark extract or any mixture thereof.

In the context of the present invention, it has surprisingly been shown that the bark of species of pine (Pinus spp.) or elm (Ulmus spp.) or extracts of such barks have a positive and also prophylactic effect on the occurrence of acidosis. This physiological effect is due, in particular, to the oligomeric procyanidins (which are also known as proanthocyanidins) contained in said barks. An inhibiting effect on ruminal amylases in the context of in-vitro tests with ruminal fluid has been detected for these substances.

In a further embodiment of the invention, the animal feed additive contains a combination of oligomeric procyanidins with iron ions and/or iron compounds, i.e. the oligomeric procyanidins are in the form of an iron-containing complex, wherein the ratio between the iron ions and/or the iron compounds and the oligomeric procyanidins is in the range 1:10 to 1:20000, preferably in the range 1:100 to 1:8000.

As an example, the animal feed additive of the invention in which the oligomeric procyanidins are present as an iron-containing complex can be manufactured as follows: a commercially available iron chloride or iron lactate solution or the like is sprayed onto ground pine bark and/or ground elm bark in the desired ratio of iron ions and iron compounds to oligomeric procyanidins and then gently dried.

In a further embodiment, the procyanidin-containing extracts are mixed in the desired ratio with said iron solutions, for example. In this regard, water-soluble procyanidin-containing extracts may also be used, which extracts form the complexes of the invention upon addition of the iron solutions. This embodiment can in particular be further processed or used in the liquid form, i.e. sprayed in the liquid form onto support materials, feedstuff premixes etc, but it may also be given to the animals in the liquid form, for example by means of a drench.

Every embodiment of the invention in which oligomeric procyanidins are complexed with iron ions and/or iron compounds has been shown to exhibit a significant improvement in the inhibiting effect on ruminal amylases in-vitro.

From a physiological viewpoint, it is important that in animals which do not develop any ruminal acidosis or which are not sensitive to it, no inhibition of the ruminal amylases occurs, since such inhibition in “healthy” animals can lead to a disruption of rumen fermentation and as a result, to health problems and to a reduction in yield. Ruminal amylase inhibitors used in the prior art (for example acarbose) are known to inhibit ruminal amylases independently of the state of health of the animals.

In the context of the invention, comprehensive series of tests were carried out wherein it was surprisingly discovered that pine bark, elm bark or extracts thereof trigger (or initiate) an action mechanism due to a drop in the pH (i.e. an increase in acidity). The oligomeric procyanidins contained in pine and elm bark are released by the drop in the pH in the range which is sensitive to the occurrence of ruminal acidosis or subacute ruminal acidosis and thus they become active. The quantity released and the rate of release can be influenced by the degree of extraction of the barks. The ground, i.e. not extracted, bark shows the largest delay in substance release and thus it has proved to be the best in acidosis-sensitive animals in avoiding a drop in the pH by inhibiting ruminal amylases without running the risk of unnecessarily perturbing rumen physiology in healthy or insensitive animals.

The expressions “acute ruminal acidosis” and “subacute ruminal acidosis” as used in this description should be understood as follows: Acute ruminal acidosis arises due to excessive fermentation activity in the rumen, with the resulting formation of acids as metabolic products of the fermentation process. The reason for this is often a large amount of feed concentrate, which is necessary during the onset of lactation in order to limit the energy deficit brought about by milk formation. A long-lasting fall in the pH in the rumen below a value of 5.5 means a change in the ruminal flora, suspension of rumination, lack of appetite, diarrhea, colic and up to severe perturbations in the general condition to downing of animals. The low pH damages the mucous membrane of the rumen and it can no longer carry out its barrier function. Transfer of bacteria, biogenic amines, fungi, fungal and bacterial toxins from the rumen into the organism is promoted and worsens the symptoms.

SARA (subacute ruminal acidosis) constitutes a widespread problem in cattle production. SARA is associated with a drop in the rumen pH below the physiological norm. The cause of rumen acidosis is usually a surfeit of rapidly fermentable carbohydrates, often in connection with a lack of bulk raw fiber. In contrast to acute ruminal acidosis, the phases of a non-physiological low pH (below 5.8) in SARA is limited in time and last from a few minutes to several hours. If these acidosis phases last longer than three or four hours, morphological damage occurs to the rumen mucous membrane which can usually only be established post mortem. This damage then in turn results in an increased permeability for bacteria, biogenic amines, fungi, fungal and bacterial toxins, which cause inflammation in the organism and encourage liver damage as well as hoof disease or can even be excreted into the milk. Consequential diseases include: hoof disease (laminitis), liver health damage (in particular abscesses), fertility problems, reduced milk production as well as reduced weight gain in beef cattle. The perturbation to the physiology of the villi in the rumen also results in a lack of desire to eat (lack of appetite) and a change in fecal consistency (i.e. feces becomes thin and watery).

Examples of conditions resulting from ruminal acidosis in a ruminant which can be treated, reduced or prevented by adding ground pine bark, ground elm bark, pine bark extract, elm bark extract or any mixture thereof, optionally treated, as described above, with an iron-containing solution, to animal feed or drinking water are the reduction of the pH in the rumen, disruption of ruminal flora, suspension of rumination, drop in milk production, drop in milk fat content, chronic metabolism problems, liver metabolism problems, infertility, longer restocking rates, fewer calves and inflammatory hoof diseases.

In accordance with a further aspect of the present invention, using the feed additive of the invention also results in reduced aflatoxin excretion in the milk of the ruminant. This reduction in aflatoxin excretion is linked to the substantially reduced frequency of acidosis due to the use of the feed additive of the invention. It arises on the one hand because the reduced acidosis frequency leads to less mucous membrane damage in the rumen so that the mucous membrane can carry out its function as a barrier better. On the other hand, the feed additive of the invention in general results in better rumen activity, which also results in decomposition of the aflatoxins.

In accordance with another aspect, the invention concerns an animal feed for ruminants which contains the animal feed additive containing the oligomeric procyanidins in a quantity which is effective in the treatment or prophylaxis of acute or subacute ruminal acidosis or conditions resulting from ruminal acidosis in a ruminant, and one or more feed components selected from the group consisting of protein carriers, carbohydrate carriers, forage, silage, fats, vitamins, minerals and trace elements.

The animal feed ration for ruminant farm livestock is basically constructed as follows:
Basic feedstuff: as a rule, agriculturally suitable feedstuff such as meadow grass and preserved grass (hay, silage), field grass and preserved field grass (alfalfa silage), by-products (for example brewer's spent grain);
Feed concentrate and supplements (yield-dependent): suitable grain, by-products from flour, sugar and oil processing as well as mineral feeds supplemented with vitamins. Currently, it is routine to use specific supplements for dairy cattle (for example milk production feed) or supplements for beef cattle (generally known as feed concentrate or beef cattle feed).

The daily feed requirements for basic feed for farm livestock are normally given in kilograms dry matter (kg DM). Thus, for dairy cows, the dry matter intake capacity is about 3% to 3.8% percentage of body weight; for fattened bulls it is 1.6% to 2.5% of the body weight. Thus, for dairy cows, the daily dry matter intake is usually 15 to 20 kilograms. The daily feed concentrate requirement for dairy cow feed concentrate (=milk production feed) is approximately 5 to 10 kilograms per animal per day; for a beef cattle feed, it is approximately 2 to 3 kilograms per animal per day.

The animal feed additive containing oligomeric procyanidins can be administered mixed with the animal feed (for example basic feed, milk production feed, feed concentrate, etc), or it can be given to the respective feed rations as a top dressing.

The animal feed of the invention contains the animal feed additive containing the oligomeric procyanidins in the form of ground pine and/or elm bark in an amount corresponding to administering 0.1 to 500 g of ground pine and/or elm bark per day per animal. Alternatively, the animal feed of the invention contains the animal feed additive containing the oligomeric procyanidins in the form of pine bark and/or elm bark extract in an amount corresponding to administering 0.002 to 100 g of pine bark extract and/or elm bark extract per day per animal. These quantities are also suitable for those embodiments in which the ground bark or extract are treated, as described above, with iron-containing solution so that the oligomeric procyanidins are present as an iron-containing complex.

The quantity of ground pine and/or elm bark used in the animal feed of the invention can be in the range 0.01 g to 250 g of ground bark per kilogram of feed dry matter. As an example, the animal feed preferably contains 0.01 g to 100 g of ground pine and/or elm bark per kilogram of milk production feed (feed concentrate for dairy cows), 0.03 g to 250 g of ground pine and/or elm bark per kilogram of beef cattle production feed (feed concentrate for beef cattle), as well as 0.03 g to 250 g of ground pine and/or elm bark per kilogram of feed concentrate for small ruminants (for example dairy sheep, meat sheep, wool sheep, dairy goats, meat goats). These quantities are the same for those embodiments in which the ground bark is treated, as described above, with an iron-containing solution so that the oligomeric procyanidins are present as iron-containing complexes.

The quantity of ground pine and/or elm bark extract used in the animal feed of the invention may be in the range 0.002 g to 50 g of bark extract per kilogram of feed dry matter. As an example, the animal feed preferably contains 0.002 g to 20 g of pine and/or elm bark extract per kilogram of milk production feed (feed concentrate for dairy cows), 0.002 g to 50 g of pine and/or elm bark extract per kilogram of beef cattle performance feed (feed concentrate for beef cattle) as well as 0.002 g to 50 g of pine and/or elm bark extract per kilogram of feed concentrate for small ruminants (for example dairy sheep, meat sheep, wool sheep, dairy goats, meat goats). These quantities are the same for those embodiments in which the extract is treated, as described above, with an iron-containing solution so that the oligomeric procyanidins are present as iron-containing complexes.

In accordance with another aspect, the invention concerns a premix for the production of an animal feed, wherein the premix contains the animal feed additive containing the oligomeric procyanidins for the treatment or prophylaxis of acute or subacute ruminal acidosis or conditions resulting from ruminal acidosis, together with one or more feedstuff components selected from the group consisting of protein carriers, carbohydrate carriers, forage, silage, fats, vitamins, minerals and trace elements.

The invention also concerns the use of an animal feed additive in accordance with the invention or a premix containing an animal feed additive in accordance with the invention, for the manufacture of an animal feed.

In accordance with another aspect, the invention concerns the use of ground pine bark, ground elm bark, pine bark extract, elm bark extract or any mixture thereof, optionally treated, as described above, with an iron-containing solution, for the manufacture of an animal feed additive or an animal feed premix or an animal feed for the treatment or prophylaxis of acute or subacute ruminal acidosis or conditions resulting from ruminal acidosis in a ruminant.

When the animal feed contains the oligomeric procyanidins in the form of ground pine bark and/or ground elm bark, the dose is preferably in the range 0.1 to 500 g of ground pine and/or elm bark per day per animal. When the animal feed contains the oligomeric procyanidins in the form of pine bark extract and/or elm bark extract, the dose is preferably in the range 0.002 to 100 g of pine bark and/or elm bark extract per day per animal. These quantities are also applicable to those embodiments in which the ground bark or the extract has been treated, as described above, with an iron-containing solution so that the oligomeric procyanidins are present as iron-containing complexes.

In accordance with a further embodiment of the invention, the pine bark and/or elm bark extract containing oligomeric procyanidins, preferably in the form of a water-soluble extract, can also be mixed with drinking water for ruminants. The quantity of bark extract in the drinking water may be in the range 0.00008 g to 100 g of extract per liter of water. Alternatively, ground pine and/or elm bark can be slaked with the drinking water. In this case, the dose is, for example, in the range 0.1 to 500 g per animal per day. These quantities are also applicable to those embodiments in which the ground bark or the extract has been treated, as described above, with an iron-containing solution so that the oligomeric procyanidins are present as iron-containing complexes.

The bark extracts can be manufactured using methods which are known per se, such as solid-liquid extraction with water, hot water or steam, or with organic solvents such as ethanol or methanol or with mixtures of solvents or mixtures of such solvents with water, or by extraction with supercritical CO₂ as the extraction medium. The degree of extraction can be optimized by hydrolytic or other catalytic decomposition conditions (for example enzymatic, in particular cellulolytic enzymes such as cellulases). Preferably, the extraction is carried out at temperatures in the range 50° C. to 300° C.

In order to optimize the procyanidin content, the bark is preferably ground and the powder obtained in this manner is extracted in a percolator with 0.1 molar dilute hydrochloric acid solution as the extraction medium at a temperature of 100° C. Next, the extract is evaporated and taken up in water.

Another preferred extraction method is supercritical CO₂ extraction, in which supercritical CO₂ is used as the extraction medium and extracted at a throughput of 1200 to 1400 liters per hour over a period of 4 hours at a pressure of 25 to 30 MPa and at a temperature or 50° C. to 70° C. The extract is then taken up in ethanol.

Standardization of the quantity of oligomeric procyanidins can be carried out using the extracts obtained with the aid of an HPLC analysis.

Other features which are considered as characteristic for the invention are set forth in the appended claims.

Although the invention is illustrated and described herein as embodied in additive for animal food or drinking water for ruminants, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1A shows the temperature and pH profiles in the rumen for an acidosis-sensitive animal;

FIG. 1B shows the temperature and pH profiles in the rumen for an acidosis-insensitive animal;

FIG. 2 shows the influence of administering an animal feed additive in accordance with the invention on the frequency of acidosis in a feed test with dairy cows;

FIG. 3 shows the influence of the animal feed additive of the invention on the rumen pH;

FIG. 4 is a bar chart illustrating a comparison between a control group and various ratios of the claimed invention; and

FIG. 5 shows a chart showing the temporal behavior of pH in rumen fluid.

### DETAILED DESCRIPTION OF THE INVENTION

### Example 1

### In-Vitro Tests with Oligomeric Procyanidins

Ruminal fluid was taken from healthy, untreated steers with rumen fistulas, centrifuged and the supernatant was immediately deep frozen. 10 mL of the thawed ruminal fluid was mixed with 0.5 g of the substance of the invention and stirred for 60 min, 90 min, 120 min, 150 min, 180 min, 210 min and 240 min at 39° C. using a magnetic stirrer. The supernatant was pipetted off at the appropriate time and the amylase activity was measured. A commercially available photometric system was used which is usually used to determine the amylase activity in blood serum was employed for this purpose.

TABLE 1
Amylase measurement in rumen fluid
using oligomeric procyanidins:

| | Rumen fluid | Rumen fluid with addition of | |
|---|---|---|---|
| | without trial | oligomeric procyanidins^{#)} | |
| Time point | substance (control) | | Amylase activity as |
| [min] | [U/l] | [U/l] | percentage of the control |
| 60 | 209 | 129 | 61.7 |
| 90 | 212 | 153 | 72.2 |
| 120 | 197 | 149 | 75.6 |
| 150 | 211 | 159 | 75.4 |
| 180 | 204 | 153 | 75.0 |
| 210 | 194 | 163 | 84.0 |
| 240 | 181 | 167 | 92.3 |

^{#)}Oligomeric procyanidins without iron ion complex

As can clearly be seen from Table 1, the oligomeric procyanidins cause an inhibition of the amylase activity.

### Example 2

### In-Vitro Tests with an Oligomeric Procyanidin-Iron Complex

Ruminal fluid was taken from healthy, untreated steers with rumen fistulas, centrifuged and the supernatant was immediately deep frozen. 10 mL of the thawed ruminal fluid was mixed with 0.5 g of the substance of the invention and stirred for 60 min, 90 min, 120 min, and 240 min at 39° C. using a magnetic stirrer. The supernatant was pipetted off at the appropriate time and the photometric amylase activity measurement was carried out.

TABLE 2

| | | Rumen fluid with | | Rumen fluid with | | Rumen fluid | |
|---|---|---|---|---|---|---|---|
| | | addition of | | addition of | | with addition of | |
| | | oligomeric | | oligomeric | | oligomeric | |
| | Rumen fluid | procyanidin iron | | procyanidin iron | | procyanidin | |
| | without trial | complex | | complex | | iron complex | |
| Time | substance | according to the | | according to the | | according to | |
| point | (control) | invention*⁾ | | invention**⁾ | | the invention***⁾ | |
| [min] | [U/I] | [U/I] | % AA | [U/I] | % AA | [U/I] | % AA |
| 0 | 333 | 333 | 100 | 333 | 100 | 333 | 100 |
| 60 | 265 | 2 | 0.8 | 4 | 1.5 | 136 | 51.3 |
| 90 | 256 | 1 | 0.4 | 2 | 0.8 | 140 | 54.7 |
| 120 | 249 | 0 | 0 | 1 | 0.4 | 146 | 58.6 |
| 240 | 241 | 0 | 0 | 0 | 0 | 144 | 59.6 |

```
  *⁾Ratio of iron ions to oligomeric procyanidins is 1:500
  **⁾Ratio of iron ions to oligomeric procyanidins is 1:10
***⁾Ratio of iron ions to oligomeric procyanidins is 1:20,000
% AA Amylase activity as a percentage of the control
```

It is clear from Table 2 that the substance of the invention, present as an iron-containing complex, causes an inhibition of the amylase activity, wherein the amylase-inhibiting action is much more pronounced than with the substance of the invention in the in-vitro test of Example 1.

### Example 3

### In-Vivo Tests with Beef Cattle

As already described above, from a physiological viewpoint it is important that in animals which do not develop any ruminal acidosis or are not sensitive to it, no inhibition of the ruminal amylases should occur. Such an inhibition in healthy animals can result in disruption of rumen fermentation which in turn has deleterious effects on health and reduction in yield.

This example shows that the test substance of the invention (for example pine bark or pine bark extract), as a function of the degree of extraction, exhibits a mechanism of action which is conditioned by a drop in the pH (i.e. an increase in acidity). Comprehensive tests have shown that the oligomeric procyanidins of pine bark, by means of an increase in acidity (i.e. a drop in pH) in a range in which the occurrence of (subacute) ruminal acidosis is likely, are released and thus become active. The released quantity and the rate of release can be influenced by the degree of extraction of the bark.

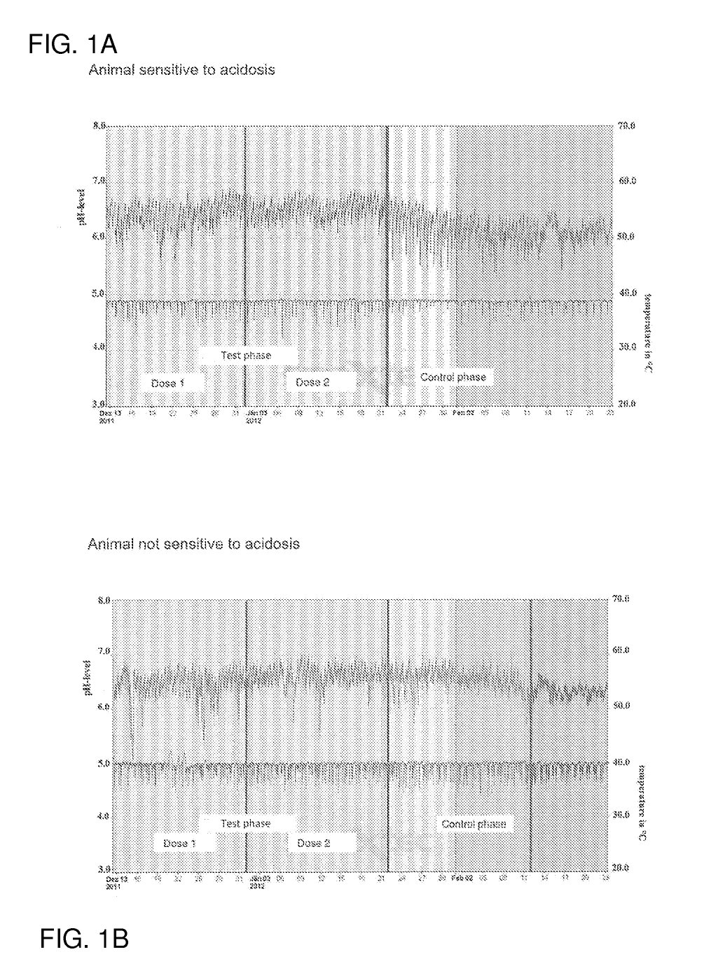

Compared with bark extracts, ground bark has the greatest delay in the release of active substance. Thus, in practice it is particularly advantageous for a drop in pH to be specifically avoided in acidosis-sensitive animals by inhibiting ruminal amylases without running the risk of unnecessarily perturbing the rumen physiology in healthy or insensitive animals (see FIGS. 1A and 1B).

These facts were obtained in comprehensive tests using rumen boli, wherein the influence of the oligomeric procyanidins was investigated in beef cattle. The rumen boli used were measuring sensors, with one sensor being placed in the rumen of each test animal. The sensor continuously measured both the pH and the temperature at intervals of a few minutes and stored these data. An external data storage station retrieved these data several times a day and fed them to a computer to analyse the data.

Fattening bulls (rather than dairy cows) were used for the tests because the determination of volatile fatty acids in dairy cows for milk formation varies as a function of the stage of lactation and the quantity of milk and thus would have been a further influencing factor.

The tests with rumen boli were carried out with six steers over a period of nine weeks in total in a Latin square test. The following were employed: on the one hand, test feed which contained oligomeric procyanidins in two different doses, as well as a control feed without admixture of an animal feed additive of the invention. The test scheme is shown in Table 3 below.

TABLE 3

| | | Test feed | Test feed |
|---|---|---|---|
| | Control feed | Dose 1 | Dose 2 |
| Period 1 (3 weeks) | Animal 1 | Animal 3 | Animal 5 |
| | Animal 2 | Animal 4 | Animal 6 |
| Period 2 (3 weeks) | Animal 5 | Animal 1 | Animal 3 |
| | Animal 6 | Animal 2 | Animal 4 |
| Period 3 (3 weeks) | Animal 3 | Animal 5 | Animal 1 |
| | Animal 4 | Animal 6 | Animal 2 |

An animal feed formed by feed concentrate, corn silage and hay was fed to the animals, wherein the feed concentrate consisted of equal parts of barley, wheat and HP soya (i.e. high protein soya extract meal). The composition of the feed is shown in Table 4 below. The animal feed additive of the invention was added to the animal feed during the test periods as a top dressing. In the case of the test feed, the animal feed additive containing oligomeric procyanidins was fed in two different doses (15 g per animal per day or 30 g per animal per day).

TABLE 4

| | | |
|---|---|---|
| Corn silage | kg | 9.09 |
| Feed concentrate | kg | 2.73 |
| Hay | kg | 0.69 |

Feed concentrate composition: 33.3% barley, 33.3% wheat, 33.3% HP soy

FIG. 1 shows the profile of the pH measured with the rumen boli and the temperature in the rumen, for an acidosis-sensitive animal (FIG. 1A) and for an acidosis-insensitive animal (FIG. 1B).

For the acidosis-sensitive animal, provocation feeding with the starch-rich animal feed in the control phase, i.e. without administration of the animal feed additive of the invention, caused a drop in the pH. In contrast, during the test phase, i.e. during the period when the animal feed additive of the invention was administered, the pH values were higher. This confirms the effectiveness of the animal feed additive of the invention in the treatment or prophylaxis of ruminal acidosis.

In the case of the acidosis-insensitive animal, in the control phase, i.e. without the animal feed additive of the invention, no drop in the rumen pH was observed. No disturbance to the rumen physiology in the test phase could be observed. This result confirms the mechanism of action described above for the animal feed additive of the invention. In healthy or acidosis-insensitive animals, the rumen physiology is not perturbed by the animal feed additive of the invention, since the drop in the pH in the rumen required to trigger its action does not occur.

### Example 4

### In-Vivo Tests with Dairy Cows

This test was a field test in a dairy farm. During the test phase, all of the lactating dairy cows of the herd received 30 g of elm bark as a top dressing in addition to the usual stall ration. The data for the usual routine stall tests as regards the health of the herd during the test phase were compared with earlier data which were deemed to be control data (without the addition of bark). The test animals were 40 lactating dairy cows. The animal feed which was administered was the feed mentioned in Example 4. The dose for the animal feed additive of the invention was 30 g of elm bark per animal per day.

In the context of this test with dairy cows, it was able to be shown that the animal feed additive of the invention resulted in a higher milk production, in particular in critical phases of lactation. As can clearly be seen in Table 5 below, this effect was surprisingly not linked to the reduction in the milk fat content anticipated from a professional viewpoint. The effect of the animal feed additive of the invention is thus in stark contrast to the teaching of the prior art, which assumes that an increase in milk production is always associated with a drop in the milk fat content.

TABLE 5
Quantity of milk and milk fat content in animals prone to acidosis

| | Routine herd examination | | |
|---|---|---|---|
| | Time 1 | Time 2 | Time 3 |
| | control | control | test |
| Number of cows over 35 kg | 16 | 16 | 11 |
| Milk production | | | |
| Quantity of milk in kg | 39.9 | 38.9 | 41.3 |
| Milk fat content, % | 4.21 | 4.23 | 4.26 |
| Number of cows in first lactation | 3 | 4 | 4 |
| in lactation day 1 to 100 | | | |
| Quantity of milk in kg | 23.2 | 29.5 | 33.6 |
| Milk fat content, % | 4.71 | 4.23 | 4.33 |
| Number of cows from second lactation | 9 | 10 | 6 |
| in lactation day 1 to 100 | | | |
| Quantity of milk in kg | 41.0 | 39.8 | 43.3 |
| Milk fat content, % | 4.06 | 4.11 | 4.26 |

The routinely obtained parameters regarding herd health are also interesting: the urea content of the milk acts as an indicator of the protein supply. The protein content of the milk is an indicator of the energy supply to the animal. The two parameters are assessed together as urea/protein and are divided into nine classes. The value should be in the central field (class 5). Deviations mean an over- or under-supply of protein and/or energy.

Metabolic urea/protein check (urea class, UCI) without animal feed additive of the invention:

A maximum 47.5% of the animals were categorized into the optimal class 5, up to 42.5% of the animals were in the unsatisfactory classes 2 (lack of energy) or 7, 8 and 9 (excessive protein and/or excessive energy).

Metabolic urea/protein check (UCI) with animal feed additive of the invention: 70.7% of the animals were categorized into the optimal class 5, 9.8% into class 2, 17.1% into class 8, no animals were in class 9.

The metabolic check energy supply parameter describes the energy supply to the animal via the protein content of the milk. A protein content of 3.2% to 3.8% is classified as normal, above it is an over-supply of energy, below it is an under-supply of energy. In principle, a lack of energy is accompanied by a drop in the milk protein content.

Metabolic check energy supply without animal feed additive of the invention: about 58% of the animals were in the “normal” range, up to 35% of the animals were in the “over-supply of energy” zone.

Metabolic check energy supply with animal feed additive of the invention: about 73% of the animals were in the “normal” range, only 17% of the animals were in the “over-supply of energy” zone.

The significant improvement in these parameters, which reflect the health of the rumen and the metabolism, indicates that the animal feed or drinking water additive of the invention can actually result in an improvement in the physiology of the rumen and thus of the entire metabolic situation in the animal.

### Example 5

### Animal Feed of the Invention for Dairy Cows

This example provides the composition of a feed concentrate for dairy cows.

TABLE 6

| Raw material | Dry matter | Fraction, % |
|---|---|---|
| Corn | 880 | 10.00 |
| Barley | 870 | 15.00 |
| Wheat | 870 | 11.50 |
| Soya meal | 870 | 10.00 |
| Corn feed concentrate | 880 | 5.00 |
| Rape meal | 886 | 15.00 |
| Wheat feed meal | 882 | 5.00 |
| Wheat bran | 880 | 10.00 |
| Dried cuttings | 906 | 15.00 |
| Feeding lime | 980 | 1.20 |
| Pine or elm bark | 920 | 0.30 |
| Mixture of minerals | 900 | 2.00 |
| | | 100.00 |

TABLE 7

| Nutrient | Unit | Content |
|---|---|---|
| Dry matter | % | 88.34 |
| Net energy - lactation (NEL) | MJ | 6.59 |
| ME-ruminants*) | MJ | 10.50 |
| Raw protein | % | 17.72 |
| Raw fiber | % | 8.14 |
| Calcium | % | 0.89 |
| Phosphorus | % | 0.60 |
| Sodium | % | 0.36 |
| Magnesium | % | 0.35 |
| Ca:P | | =1.49:1 |
| Milk from protein | l | 2.09 |
| Milk from NEL | l | 2.10 |

*)Metabolic energy: energy rating scheme for ruminants

### Example 6

### Animal Feed in Accordance with the Invention for Beef Cattle

This example provides a composition for a feed concentrate for beef cattle.

TABLE 8

| Raw material | Dry matter | Fraction, % |
|---|---|---|
| Barley | 870 | 9.80 |
| Wheat | 870 | 10.00 |
| Soya meal | 870 | 19.00 |
| Corn feed concentrate | 880 | 5.00 |
| Rape meal | 886 | 20.00 |
| Wheat feed meal | 882 | 5.00 |
| Wheat bran | 880 | 10.00 |
| Dried cuttings | 906 | 15.00 |
| Feeding lime | 980 | 2.00 |
| Cattle lick | 900 | 0.20 |
| Pine or elm bark | 920 | 1.00 |
| Mixture of minerals | 900 | 3.00 |
| | | 100.00 |

TABLE 9

| Nutrient | Unit | Content |
|---|---|---|
| Dry matter | % | 88.39 |
| ME-ruminants*) | MJ | 10.29 |
| Raw protein | % | 22.01 |
| Raw fiber | % | 8.90 |
| Calcium | % | 1.74 |
| Phosphorus | % | 0.76 |
| Calcium:phosphorus | | = 2.29:1 |
| Sodium | % | 0.31 |
| Magnesium | % | 0.44 |
| Vitamin D | IE | 3900 |
| Vitamin E | mg | 19 |
| Vitamin B1 | mg | 1.56 |
| ME-R*)/kg dry matter | MJ | 11.65 |
| % Raw fiber/kg dry matter | | 10.07 |

*)Metabolic energy: energy rating scheme for ruminants

In principle, a feed of this type is also suitable for small ruminants such as sheep in milk, meat and wool production and for goats in milk and meat production.

### Example 7

### In-Vivo Test with Dairy Cows

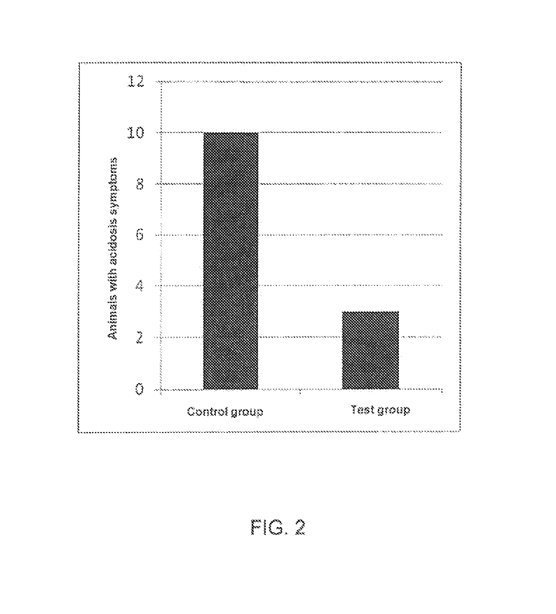

In the context of a further feed test with lactating dairy cows, the substance of the invention in the embodiment of an iron-containing complex with a ratio of iron to oligomeric procyanidins of 1:500 was administered in a dose of 25 g per animal per day. In total, 54 lactating dairy cows (breed: German Holstein) took part in this test. The 27 animals of the test group obtained the substance of the invention in the given dosages over a period of 14 days via the milk production feed. The control group was again constituted by 27 animals and was fed with the same milk production feed without adding the substance of the invention. The acidosis frequency in the animals was measured using clinical parameters and using metabolic parameters. As can be seen in FIG. 2, a reduction of 70% in the frequency of acidosis was measured in the test group. This constitutes a further optimization of the effects of the substance of the invention as a combination of iron ions or iron compounds with the oligomeric procyanidins; this effect could be obtained with a dose reduced by approximately 20%.

### Example 8

### Influence of the Novel Animal Feed Additive on Rumen pH

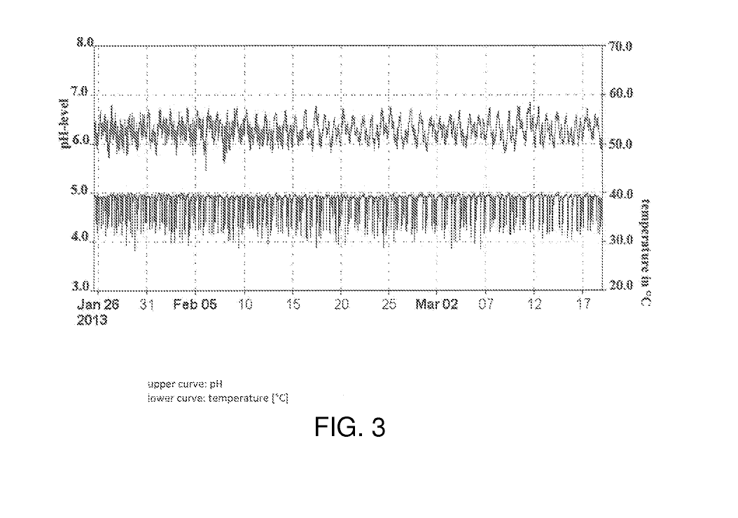

A further particular advantage of the substance of the invention, present as a complex of iron ions or iron compounds with oligomeric procyanidins, is the fact that small variations in the rumen pH can be smoothed out over the course of the day. This emerges from the measurement protocol shown in FIG. 3, in which the effect of the substance of the invention on rumen pH was examined. After administration of the test substance of the invention from Example 7 (after 15 February) in a dose of 25 g per animal per day, in the measurement protocol a much smaller variation in the individual daily variations in the pH was observed. Larger variations in these daily variations are assumed to be a major damaging factor for the health of the ruminal mucous membrane and, furthermore, for the sensitivity of an animal to subclinical acidosis.

### Example 9

### Influence of the Animal Feed Additive of the Invention on the Excretion of Aflatoxin in Milk

In the context of a feed test in a dairy farm, the influence of the substance of the invention on the excretion of aflatoxins into milk was examined. In the context of the test, the farm animals were divided into a control group and a test group, each with 50 lactating dairy cows. The test group's feed was supplemented for a period of 14 days by means of the milk production feed using the substance of the invention in a dose of 30 g per animal per day. At the end of the test period, the aflatoxin content of the milk samples in both groups was measured; in the pooled milk samples from the control group, the aflatoxin content was 39.2 ppt (parts per trillion), and in the combined milk samples from the test group, it was 11 ppt. This constitutes a reduction of 72 percent.

TABLE 10
Aflatoxin test 1

Aflatoxin content in ppt

| Control group | Test group | Reduction, % |
|---|---|---|
| 39.2 | 11.0 | 72 |

Furthermore, in this feed test, the parameters for the risk of acidosis were assessed and it was established that a significant improvement to the frequency of acidosis and the resulting metabolic parameters occurred. Thus, the relevant for acidosis to occur, namely the fat-protein quotient, could be raised in the test group. This confirms the direct relationship between the reduction in the frequency of acidosis and the resulting improved decomposition of aflatoxins in the rumen or the resulting reduced excretion of aflatoxins into the milk.

In the context of a further feed test with lactating dairy cows, the substance of the invention, in the form of an iron-containing complex (Fe to oligomeric procyanidins ratio 1:350), was used in dose of 25 g per animal per day. This test was carried out with a total of 72 lactating dairy cows over a period of 21 days. The 36 animals of the test group received the substance of the invention in the given dose throughout the test period by means of the milk production feed. At the end of the test period, the aflatoxin content in the pooled milk samples from the groups was measured. In the control group, the measured value was 75.2 ppt; in the test group, it was 19.6 ppt. This constitutes a reduction of 74 percent.

TABLE 11
Aflatoxin test 2

Aflatoxin content in ppt

| Control group | Test group | Reduction, % |
|---|---|---|
| 75.2 | 19.6 | 74 |

### Example 10

### In Vivo Trial with Dairy Cows

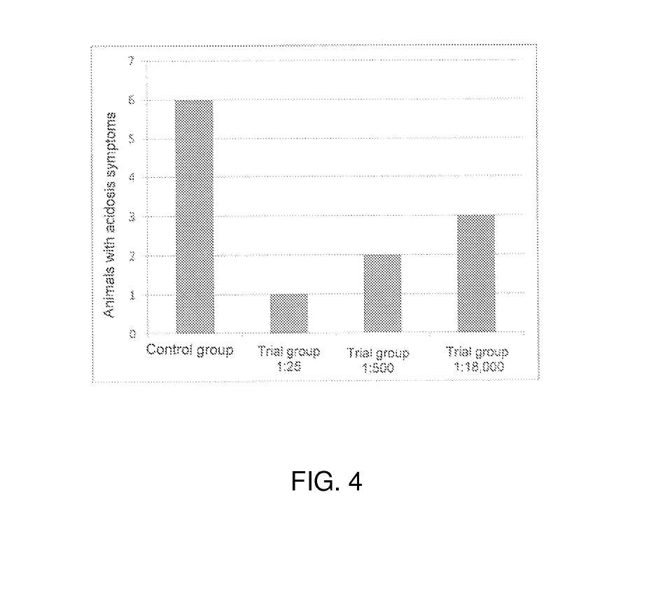

In the course of a feeding trial with lactating dairy cows, the inventive substance, embodied as an iron-containing complex, was administered at a dosage of 25 g per animal per day. 52 lactating cows (breed: German Holstein) were included in the trial. A control group was made up of 13 animals, the other 39 were divided into three trial groups of 13 animals each. The feeding regimen differed between trial groups in that the trial substances were present in different embodiments. The ratio of iron to oligomeric procyanidins varied from group to group (1:25; 1:500; 1:18,000). The animals in the trial groups received the inventive substances in the indicated dosages over a period of 21 days via the milk production feed. The control group was fed with the same milk production feed without the addition of the inventive substance. The incidence of acidosis among the animals was measured using clinical parameters and with reference to metabolism parameters. In the trial groups, the result from Example 7 was confirmed, according to which the incidence of acidosis fell significantly (FIG. 4). Thus, it is demonstrated that the substance according to the invention embodied as a combination of iron ions or iron compounds with oligomeric procyanidins is effective in the described ratio band.

### Example 11

### In Vivo Trial with Dairy Cows

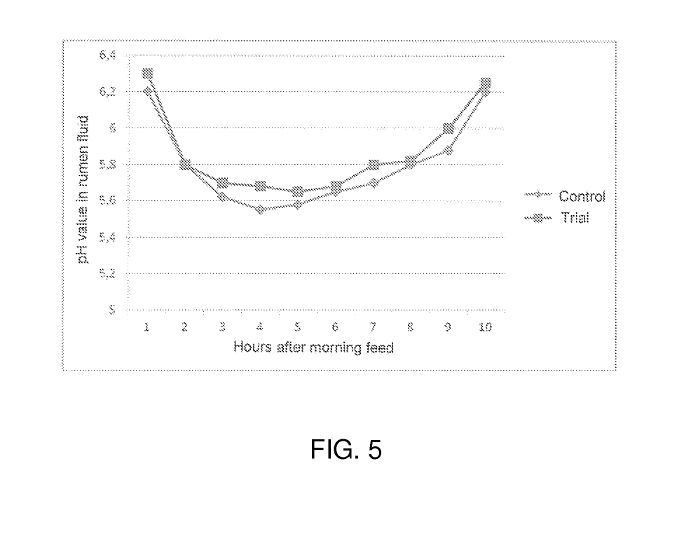

In the course of a feeding trial with lactating dairy cows, the inventive substance, embodied as an iron-containing complex, was administered at a dosage of 25 g per animal per day. 10 lactating dairy cows were included in the trial. The control group consisted of 5 animals, and the trial group also included 5 animals. Both groups received the standard feed ration, which consisted of corn silage, hay and milk production feed. The trial group was given the trial substance embodied as an iron-containing complex in a ratio of 1:25 iron to oligomeric procyanidins in a dose of 25 g on-top to the milk production feed. The trial lasted for 14 days. All animals were provided with a rumen fluid bolus for automatic, continuous recording of the pH value in the rumen fluid. FIG. 5: The pH curves of the two groups were compared over the course of the day starting with the morning feed and ending with the evening feed. The measured low pH value was significantly higher in the trial group than in the control group. As the figure also shows, the pH values in the trial did not fall below 5.6 throughout the entire duration of the trial, whereas they fell below this value for about 1.5 hours in the control group. This trial confirms that the inventive substance is effective in stabilising the pH value in the rumen fluid in its present form and at the dosage used.

### Example 12

In the course of extensive feeding trials with lactating dairy cows, the effect of an inventive substance in the form of ground pine bark in various ratios of iron ions to oligomeric procyanidins and in different dosages was investigated with regard to the aflatoxin content in milk. The inventive substance was fed to an average of 68 animals in four different dairy cattle operations (an average of 34 animals in the control group and 34 animals in the trial group at each facility) in the form of ground pine bark in various ratios of iron ions to oligomeric procyanidins. Each test cycle lasted 14 days, during which the inventive substance was fed at a defined dosage on top of the milk production feed. Thereafter, the animals received no trial substance for 7 days. Then, the trial substance was administered for a further 14 days at a different dosage on top of the milk production feed. The aflatoxin contents were measured continuously in aggregate samples from the groups. In each case, the aflatoxin contents of the control groups were compared with those of the trial groups at the same facility throughout the 14-day trial cycles as average values of all collected data of a cycle, and on this basis one value for the trial group and one value for the control group for each ratio of iron ions to oligomeric procyanidins and dosage was calculated and compared.

TABLE 12
The percentage values show the average reduction in
aflatoxin content in the milk between the trial group
and the control group at the various facilities.

| | Ratio of iron | Dosage delivered on top | | |
|---|---|---|---|---|
| | ions to oligomeric | of milk production feed | | |
| | procyanidins | 5 g | 25 g | 500 g |
| Facility A | 1:10 | 52% | 64% | 67% |
| Facility B | 1:50 | 56% | 70% | 76% |
| Facility C | 1:500 | 63% | 75% | 77% |
| Facility D | 1:18,000 | 68% | 77% | 80% |

The table shows that the reduction in the aflatoxin content in milk is in a range from 50% to 80%.

1. An animal feed additive for use in the treatment or prophylaxis of acute or subacute ruminal acidosis or conditions resulting from ruminal acidosis in a ruminant, the additive comprising oligomeric procyanidins complexed with iron ions and/or iron compounds, wherein a ratio between the iron ions and/or iron compounds and the oligomeric procyanidins lies in a range from 1:10 to 1:20000.

2. The animal feed additive according to claim 1, wherein the ratio lies in a range from 1:100 to 1:8000.

3. The animal feed additive according to claim 1, wherein said oligomeric procyanidins are selected from the group consisting of ground pine bark, pine bark extract, and any mixture thereof.

4. An animal feed for ruminants, comprising:
an animal feed additive according to claim 1 in an effective quantity for the treatment or prophylaxis of ruminal acidosis or conditions resulting from ruminal acidosis; and
one or more feed material components selected from the group consisting of protein carriers, carbohydrate carriers, forage, silage, fats, vitamins, minerals and trace elements.

5. The animal feed according to claim 4, which comprises ground pine bark which has been treated with an iron-containing solution in an amount of 0.01 g to 250 g per kilogram of feed dry matter.

6. The animal feed according to claim 4, which comprises pine bark extract which has been treated with an iron-containing solution in an amount of 0.002 g to 50 g per kilogram of feed dry matter.

7. A premix for the manufacture of an animal feed for the treatment or prophylaxis of ruminal acidosis or conditions resulting from ruminal acidosis, comprising the animal feed additive according to claim 1 together with one or more feed components selected from the group consisting of protein carriers, carbohydrate carriers, forage, silage, fats, vitamins, minerals and trace elements.

8. A composition of matter, comprising:
ground pine bark, pine bark extract or any mixture thereof which has been treated with an iron-containing solution containing iron ions and/or iron compounds;
wherein a ratio between the iron ions and/or iron compounds and oligomeric procyanidins contained in said pine bark and/or in said pine bark extract lies in a range from 1:10 to 1:20000;
formulated for the treatment or prophylaxis of acute or subacute ruminal acidosis or conditions resulting from ruminal acidosis in a ruminant.

9. The composition according to claim 8, wherein the ratio lies between 1:100 and 1:8000.

10. A method of treating or prophylactically treating acute or subacute ruminal acidosis or conditions resulting from ruminal acidosis in a ruminant, the method which comprises feeding the ruminant 0.1 to 500 g per day of the composition according to claim 8 in the form of ground pine bark which has been treated with an iron-containing solution.

11. The method according to claim 10, wherein conditions resulting from ruminal acidosis in the ruminant are selected from the group consisting of a reduction in the pH in the rumen, disruption of the ruminal flora, suspension of rumination, a drop in milk production, a drop in milk fat content, chronic metabolic problems, liver metabolism problems, infertility, a longer re-stocking rate, a smaller number of calves and inflammatory hoof disease.

12. A method of treating or prophylactically treating acute or subacute ruminal acidosis or conditions resulting from ruminal acidosis in a ruminant, the method which comprises feeding the ruminant 0.002 to 100 g per day of the composition according to claim 8 in the form of pine bark extract which has been treated with an iron-containing solution.

13. The method according to claim 12, wherein conditions resulting from ruminal acidosis in the ruminant are selected from the group consisting of a reduction in the pH in the rumen, disruption of the ruminal flora, suspension of rumination, a drop in milk production, a drop in milk fat content, chronic metabolic problems, liver metabolism problems, infertility, a longer re-stocking rate, a smaller number of calves and inflammatory hoof disease.

14. A method of treating or prophylactically treating acute or subacute ruminal acidosis or conditions resulting from ruminal acidosis in a ruminant, the method which comprises feeding the ruminant the composition according to claim 8 in the form of ground pine bark treated with an iron-containing solution, pine bark extract treated with an iron-containing solution, or any mixture thereof as a drinking water additive.

15. The method according to claim 14, wherein the drinking water additive contains 0.00008 g to 100 g per liter of pine bark extract which has been treated with an iron-containing solution.

16. The method according to claim 14, wherein conditions resulting from ruminal acidosis in the ruminant are selected from the group consisting of a reduction in the pH in the rumen, disruption of the ruminal flora, suspension of rumination, a drop in milk production, a drop in milk fat content, chronic metabolic problems, liver metabolism problems, infertility, a longer re-stocking rate, a smaller number of calves and inflammatory hoof disease.

17. A method of reducing aflatoxin excretion in the milk of a ruminant, which comprises feeding the ruminant ground pine bark, pine bark extract or any mixture thereof, which has been treated with an iron-containing solution, wherein the ratio between the iron ions and/or iron compounds and oligomeric procyanidins contained in the pine bark and/or in the pine bark extract lies in a range from 1:10 to 1:20000.

18. The method according to claim 17, wherein the range is from 1:100 to 1:8000.

19. A composition for reducing aflatoxin excretion in the milk of a ruminant, comprising oligomeric procyanidins which have been complexed with iron ions and/or iron compounds, wherein a ratio between iron ions and/or iron compounds and the oligomeric procyanidins lies in a range from 1:10 to 1:20000.

20. The composition according to claim 19, wherein the range is from 1:100 to 1:8000.
